(19) United States
(12) Patent Application Publication
(10) Pub. No.: US 2011/0245922 A1
(43) Pub. Date: Oct. 6, 2011

(54) MATERIAL FOR FILLING BONE DEFECTS AND PRODUCTION METHOD THEREOF

(75) Inventors: Toshihiro KASUGA, Kiyosu (JP); Hiroki OZASA, Nagoya (JP); Akiko OBATA, Nagoya-shi (JP)

(73) Assignee: NATIONAL UNIVERSITY CORPORATION NAGOYA INSTITUTE OF TECHNOLOGY, Nagoya-shi (JP)

(21) Appl. No.: 13/074,790

(22) Filed: Mar. 29, 2011

(30) Foreign Application Priority Data
Mar. 31, 2010 (JP) 2010-080139

(51) Int. Cl. A61F 2/28; D01D 5/00

(52) U.S. Cl. 623/16.11; 264/465

(57) ABSTRACT

A material for filling bone defects has a flocculent three-dimensional structure composed of a fibrous substance containing a biodegradable resin as a principal component and further containing a siloxane. The material is produced by dissolving or suspending a substance in a solvent to give a solution or slurry of the substance, the substance containing the biodegradable resin as a principal component and further containing the siloxane, the solution or slurry having such a viscosity as to form a fibrous substance having an average diameter of 10 μm or more; and carrying out electrospinning of the solution or slurry, in which the electrospinning is performed with air blowing. The flocculent three-dimensional structure is expected to show high cell invasion efficiency, because a two-dimensionally structured nonwoven fabric shows high cell invasion efficiency when it has an average diameter of 10 μm or more.

### FIELD OF THE INVENTION

The present invention relates to bioactive materials which are useful as bone-repairing materials for filling bone defects and are used in the fields of oral or maxillofacial surgery and orthopedic surgery. More specifically, the present invention relates to a material for filling bone defects, which material has a three-dimensional structure including, as its skeleton, a composite fiber with a bioresorbable-biodegradable resin. Such a bioresorbable-biodegradable resin helps to improve the affinity for the bone and can be absorbed in vivo. The present invention also relates to a method for producing the material for filling bone defects.

### RELATED ART OF THE INVENTION

Some materials, when buried or implanted in bone defects, react with the bone and are directly chemically combined with the bone. These materials are called bioactive materials and are further classified into superficial bioactive materials which undergo a reaction only in the surface thereof; and bioresorbable materials which undergo a reaction not only in the surface but also the inside thereof and are gradually replaced with the bone. Exemplary commercialized superficial bioactive materials include hydroxyapatite ceramics (e.g., trade name APACERAM supplied by HOYA CORPORATION, Japan); and exemplary commercialized bioresorbable materials include beta phase tricalcium phosphate ceramics (e.g., trade name OSferion supplied by Olympus Terumo Biomaterials Corp., Japan).

Calcium carbonate (CaCO₃) and gypsum (CaSO₄′2H₂O) are also known as bioresorbable materials. These substances, however, have low strength and toughness and are difficult to be machined. In contrast, biodegradable polymers such as poly(lactic acid)s, poly(glycolic acid)s, copolymers of them, and polycaprolactones are highly flexible and are easy to be machined. The biodegradable polymers, however, do not show osteogenic ability (bone forming ability) because their biodegradability is derived from the phenomenon that they are degraded in vivo and are egested. In addition, there have been some reports that some of the biodegradable polymers may affect surrounding tissues because they are degraded typically into lactic acid or glycolic acid upon degradation and thus show acidity. Under such circumstances, there have been made investigations to provide composite materials between these inorganic compounds and organic compounds to allow the composite materials to have both osteogenic ability and bioresorbability and further have improved mechanical properties. Typically, Japanese Unexamined Patent Application Publication (JP-A) No. 2001-294673 discloses a process for the preparation of a bioresorbable material by combining a poly(lactic acid) and a calcium carbonate. Specifically, this literature refers to a process for synthesizing a bioresorbable material by mixing a calcium carbonate containing vaterite as a principal component with a biodegradable polymer compound such as a poly(lactic acid), which vaterite is highly soluble in water among such calcium carbonates. This technique is also advantageous in that the pH is always maintained around neutrality, because even when the poly(lactic acid) is decomposed to be acidic, the acidity is neutralized by the buffering effects of the dissolved calcium carbonate.

In this super-graying society, bone defects should be desirably cured as soon as possible, because it is very important for the health maintenance to maintain and ensure mastication and exercise performance. To improve osteogenic ability, there have been attempted to incorporate, to a bioresorbable membrane, a factor such as a bone-formation inducer (see Japanese Unexamined Patent Application Publication (JP-A) No. H06 (1994)-319794), or a proliferation factor or bone morphogenic protein (see Japanese Unexamined Patent Application Publication (Translation of PCT Application) (JP-A) No. 2001-519210; and Japanese Unexamined Patent Application Publication (JP-A) No. 2006-187303). However, these factors are hard to manage. Accordingly, demands have been made to develop a bioresorbable material having superior bone reconstruction ability to allow more reliable and more rapid self-regeneration of the bone.

In view of recent trends of researches and technologies for bio-related materials, the main stream of researches has been shifted from a materials design for the bonding of a material with the bone to a materials design for the regeneration of the bone; in these researches, the role of silicon in bone formation has been received attention; and there have been designed a variety of silicon-doped materials (TSURU Kanji, OGAWA Tetsuro, and OGUSHI Hajime, “Recent Trends of Bioceramics Research, Technology and Standardization,” Ceramics Japan, 41, 549-553 (2006)). For example, there has been reported that the controlled release of silicon genetically acts on cells to promote bone formation (H. Maeda, T. Kasuga, and L. L. Hench, “Preparation of Poly(L-lactic acid)-Polysiloxane-Calcium Carbonate Hybrid Membranes for Guided Bone Regeneration,” Biomaterials, 27, 1216-1222 (2006)). Independently, when composites of a poly(lactic acid) with one of three calcium carbonates (calcite, aragonite, and vaterite) are prepared and soaked in a simulated body fluid (SBF), the composite of the poly(lactic acid) with vaterite forms a hydroxyapatite having bone-like composition and dimensions within a shortest time among the three composites (H. Maeda, T. Kasuga, M. Nogami, and Y. Ota, “Preparation of Calcium Carbonate Composite and Their Apatite-Forming Ability in Simulated Body Fluid,” J. Ceram. Soc. Japan, 112, 5804-808 (2004)). These findings demonstrate that the use of vaterite which gradually releases silicon is believed to be a key to provide a material that gives more rapid bone reconstruction.

To use a material for filling bone defects, the affected area (bone defect) is incised, and a dense or porous material having such dimensions as to fill the affected area sufficiently is directly implanted, or a granular material is charged into the affected area.

For ensuring bone formation, it is desirable to implant or bury such a material in the affected area without a gap (clearance). However, it is not easy to process a dense or porous material so as to fit the dimensions of the affected area; and a granular material, if charged, often drops off from the affected area after the surgery (implantation). These techniques therefore have room for improvements.

Independently, though being not a technique of charging such a material into the affected area, there is also known a guided bone regeneration technique of using a masking membrane to cover the bone defect. The masking membrane has the functions of preventing the invasion of cells and tissues, which are not involved in bone formation, into the bone defect, allowing the self-regeneration ability of the bone to exhibit, and helping the bone to reconstruct. This technique is intended to cure, the bone defect by using the curing ability which a living body inherently has. For example, Japanese Unexamined Patent Application Publication (JP-A) No. 2009-61109 discloses a guided bone regeneration membrane and a production method thereof, which guided bone regeneration membrane has a bilayer structure including a first nonwoven fabric layer and a second nonwoven fabric layer, in which the first nonwoven fabric layer contains a silicon-releasable calcium carbonate and a biodegradable resin as principal components, and the second nonwoven fabric layer contains a biodegradable resin as a principal component. It has been reported that the use of this membrane gives satisfactory proliferation of murine osteoblast-like cells (MC3T3-E1 cells), and when a bone defect formed in a rabbit cranial bone is covered by the membrane, satisfactory bone formation (osteogenesis) is observed in the membrane (see T. Wakita, A. Obata and T. Kasuga, “New Fabrication Process of Layered Membranes Based on Poly(Lactic Acid) Fibers for Guided Bone Regeneration,” Materials Transactions, 50[7], 1737-1741 (2009)). This membrane, however, is not usable as a material for filling bone defects because of having a small thickness of from 230 to 300 μm.

### SUMMARY OF THE INVENTION

Accordingly, there has been a demand to provide a bioresorbable material for filling bone defects, which material has a controlled release system of such a chemical composition as to guide bone reconstruction ability effectively and has a three-dimensional structure having such a flexibility as to fit in an affected area satisfactorily.

Under these circumstances, the present inventors devised a material for filling bone defects and a production method thereof and filed a patent application as Japanese Patent Application No. 2009-163320, the entire contents of which are incorporated herein by reference. The material for filling bone defects has a flocculent three-dimensional structure composed of a fibrous substance containing a biodegradable resin as a principal component and further containing a siloxane. This material is produced by dissolving or suspending a substance in a solvent to give a solution or slurry, the substance containing the biodegradable resin as a principal component and bearing the siloxane; adding water to the solution or slurry to give a spinning solution, the water having a relative dielectric constant larger than that of the biodegradable resin; subjecting the spinning solution to electrospinning while applying a positive charge to a collector by a voltage supply and grounding a nozzle of a syringe without applying a charge thereto; thereby forming the material on the collector.

In addition, the present inventors have experimentally found that, through not being a three-dimensional structure, the pore size of a nonwoven fabric as described in JP-A No. 2009-61109 depends on the diameter of a fibrous substance constituting the nonwoven fabric; and that the fibrous substance, when having an average diameter of 10 μm or more, shows higher cell invasion efficiency than one having an average diameter of less than 10 μm. This indicates that the fibrous substance preferably has an average diameter of 10 μm or more for the invasion, growth, and proliferation of cells within the nonwoven fabric.

Based on this, it is expected that a flocculent material for filling bone defects shows high cell invasion efficiency by having an average diameter of its fibrous substance of 10 μm or more. However, the method for forming the material for filling bone defects disclosed in Japanese Patent Application No. 2009-163320 fails to form a flocculent material for filling bone defects composed of a fibrous substance having an average diameter of 10 μm or more.

Accordingly, an object of the present invention is to provide a bioresorbable material for filling bone defects, which has a controlled release system with such a chemical composition as to guide bone reconstruction ability effectively, which has a flocculent three-dimensional structure having such a flexibility as to fit in an affected area satisfactorily, and which is expected to show high cell invasion efficiency. Another object of the present invention is to provide a production method of the material.

To achieve the objects, the present invention provides, in one aspect, a method for producing a material for filling bone defects, the material having a flocculent three-dimensional structure composed of a fibrous substance containing a biodegradable resin as a principal component and further containing a siloxane, the method comprising the steps of dissolving or suspending a substance in a solvent to give a solution or slurry of the substance, the substance containing the biodegradable resin as a principal component and further containing or bearing the siloxane, the solution or slurry having such a viscosity as to form a fibrous substance having an average diameter of 10 μm or more; and carrying out electrospinning of the solution or slurry, in which the electrospinning is performed with air blowing.

When a solution or slurry of a substance is prepared by dissolving or suspending the substance in a solvent, the substance containing a biodegradable resin as a principal component and further containing or bearing a siloxane, the solution or slurry having such a controlled viscosity as to form a fibrous substance having an average diameter of 10 μm or more, and electrospinning of the solution or slurry is carried out, the solution or slurry is jetted from a nozzle to a collector, and the jetted solution or slurry is elongated and thereby fiberized by the action of an electric field to deposit a fibrous substance containing the biodegradable resin as a principal component and further containing the siloxane on the collector. The fibrous substance deposited on the collector, if still containing the solvent, becomes softened, lies on top of one another, is thereby two-dimensionally deposited to form a nonwoven fabric.

In contrast, according to the present invention, the spinning (electrospinning) is carried out with air blowing to enhance the evaporation of the solvent so that the fibrous substance can reach the collector while containing substantially no solvent. The fibrous substance deposited on the collector therefore contains substantially no solvent, thereby does not become softened, and maintains its fibrous shape. This allows the fibrous substance to be deposited three-dimensionally without lying on the top of one another and gives a flocculent three-dimensional structure composed of a fibrous substance having an average diameter of 10 μm or more. The resulting material for filling bone defects according to the present invention is expected to show high cell invasion efficiency due to the fibrous substance having an average diameter of 10 μm or more.

The present invention further provides, in another aspect, a material for filling bone defects, the material having a flocculent three-dimensional structure comprising a fibrous substance containing a biodegradable resin as a principal component and further containing a siloxane in which the fibrous substance has an average diameter of 10 μm or more. The material for filling bone defects is produced by the method according to the present invention. The thus-produced material for filling bone defects is expected to show high cell invasion efficiency due to the fibrous substance having an average diameter of 10 μm or more.

If the fibrous substance has an average diameter of more than 100 μm, the material for filling bone defects has voids of a size of several hundred micrometers or more. This impedes the supporting of cells in voids by the fibrous substance (fibers) constituting or surrounding the voids but limits the cells to being present only on the surface of the fibrous substance. To avoid this, the fibrous substance preferably has an average diameter of 100 μm or less as herein. From the viewpoint of allowing the resulting material to be decomposed at an early stage typically of shorter than three months, the fibrous substance more preferably has an average diameter of 50 μm or less. This is because it takes a long time for the fibrous substance to be decomposed in vivo if the fibrous substance is too thick.

### BRIEF DESCRIPTION OF THE DRAWINGS

FIG. 1 is a schematic diagram of an electrospinning system for use herein;

FIG. 2 is a photograph showing the appearance of the electrospinning system after spinning in Example 1; and

FIG. 3 is a scanning electron micrograph (SEM) of a fibrous substance which was prepared in Example 1 and formed a three-dimensional structure.

### DETAILED DESCRIPTION OF THE PREFERRED EMBODIMENTS

In a preferred embodiment of the present invention, spinning is carried out through electrospinning with air blowing to produce a material for filling bone defects having a flocculent three-dimensional structure which is composed of a fibrous substance containing a biodegradable resin as a principal component and further containing a siloxane.

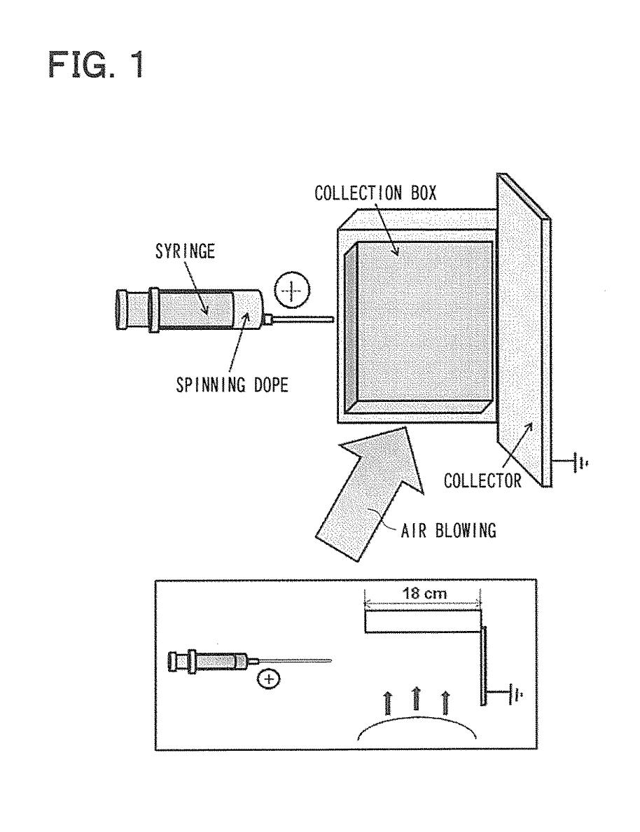

FIG. 1 depicts the schematic structure of an electrospinning system for use herein. With reference to FIG. 1, the spinning through electrospinning (herein after also briefly referred to as “electrospinning”) is carried out by applying a charge to a nozzle of a syringe from a voltage supply, i.e., by applying a positive charge to the spinning dope; and slowly extruding the dope from the tip of the nozzle. At the time when the electric field effect becomes larger than the surface tension, the dope is stretched into fibers, the fibers travel toward a collector of a grounded electrode, reach the collector while evaporating the solvent, and are deposited as a fibrous substance on the collector.

In the embodiment, the electrospinning is carried out with air blowing. Specifically, as is illustrated in FIG. 1, air is blown to the space between the nozzle and collector with a blower during the spinning. The air blowing is conducted to enhance the evaporation of the solvent.

The direction of the blowing is not limited, as long as enhancing the evaporation of the solvent. Typically, the blowing may be conducted to the space between the nozzle and the collector in a lateral direction (from the side) of the space; in a direction from the nozzle toward the collector; or in a direction from the collector toward the nozzle.

However, the blowing is preferably conducted to the space between the nozzle and the collector in a lateral direction (from the side) of the space by arranging the blower and a collection box on the both sides of the space between the nozzle and the collector, as illustrated in FIG. 1. This allows easier recovery (collecting) of the deposited flocculent fibrous substance.

The blower may be arranged so as to enhance the evaporation of the solvent and to recovery the deposited flocculent fibrous substance.

As the spinning dope, a solution or slurry of a substance in a solvent is used, which substance contains a biodegradable resin as a principal component and further contains a siloxane.

Preferred examples of the biodegradable resin include a poly (lactic acid) (PLA); and a copolymer of a poly (lactic acid) and a poly(glycolic acid) (PGA) (i.e., lactic acid-glycolic acid copolymer). Examples of biodegradable resins usable herein include synthetic polymers such as polyethylene glycols (PEGs), polycaprolactones (PCLs), PLAs, PGAs, and copolymers of PEG and PCL; and natural polymers such as fibrin, collagen, alginic acid, hyaluronic acid, chitin, and chitosan.

Exemplary solvents include chloroform and dichloromethane. Acetone may also be used when some copolymers typically of a poly(lactic acid) and a poly(glycolic acid) (PGA) are used.

The solution of a substance in a solvent, the substance containing a biodegradable resin as a present invention and further containing a siloxane may be representatively prepared in the following manner. A PLA is dissolved in chloroform (CHCl₃) and/or dichloromethane, and an aqueous solution of aminopropyltriethoxysilane (APTES) is added to give the target solution. The weight ratio of PLA:APTES (PLA to APTES) is possibly from 1:0.01 to 1:0.5, but is preferably from 1:0.01 to 1:0.05 (by weight). This is because most of APTES, if added in an excessively large amount, is dissolved out in early stages of soaking in the aqueous solution and thereby not so effective. The PLA has a molecular weight of from about 20×10⁴ to about 30×10⁴ kDa. The concentration of the PLA in the solution is preferably from 8 to 15 percent by weight for satisfactory spinning. For maintaining satisfactory spinning conditions, dimethylformamide and/or methanol may be added to the solution in a proportion of about 50 percent by weight or less relative to chloroform and/or dichloromethane.

The substance containing a biodegradable resin as a principal component and further containing or bearing a siloxane may also be prepared in the following manner. Calcium carbonate microparticles doped with a siloxane dispersed therein (Si—CaCO₃) is prepared typically by the method described in Japanese Unexamined Patent Application Publication (JP-A) No. 2008-100878; and 60 percent by weight or less of the Si—CaCO₃ microparticles is mixed with a PLA. The amount of the Si—CaCO₃ microparticles is preferably from 10 to 60 percent by weight relative to the PLA. This is because the Si—CaCO₃, if present in an amount of more than 60 percent by weight, may impede uniform admixing; and, in contrast, the Si—CaCO₃, if present in a content of less than 10 percent by weight, may not significantly exhibit its effect of controlled releasing of silicon. To uniformly disperse the microparticles, the substance is preferably prepared by kneading the PLA and Si—CaCO₃ microparticles in predetermined proportions in a heating kneader to give a composite, and dissolving the composite in the solvent to give a spinning dope.

The viscosity of the spinning dope is controlled so as to form a fibrous substance having an average diameter of 10 μm or more. This is because the higher the viscosity of the spinning dope is, the larger the diameter of the formed fibrous substance is. Specifically, the viscosity may be controlled typically by modifying the concentration of the dope and the molecular weight of PLA.

If the fibrous substance has an average diameter of more than 100 μm, the material for filling bone defects has voids of a size of several hundred micrometers or more. This impedes the supporting of cells in the voids by the fibrous substance (fibers) constituting or surrounding the voids but limits the cells to being present only on the surface of the fibrous substance. From the viewpoint of allowing the resulting material to be decomposed at an early stage typically of shorter than three months, the fibrous substance more preferably has an average diameter of 50 μm or less. This is because it takes a long time for the fibrous substance to be decomposed in vivo if the fibrous substance is too thick. For these reasons, the viscosity of the spinning dope may be controlled so as to form a fibrous substance having an average diameter of preferably 100 μm or less, and more preferably 50 μm or less.

The present inventors have experimentally found that spinning, if carried out in a draught free environment (without air blowing), does not give a flocculent three-dimensional structure but gives merely a two-dimensional nonwoven fabric composed of a two-dimensionally deposited fibrous substance, even when the spinning dope as herein is used. This is probably because the fibrous substance deposited on the collector still contains the solvent, thereby becomes softened, lies on top of one another, and is two-dimensionally deposited to form the nonwoven fabric.

In contrast, the spinning carried out with air blowing as in the embodiment of the present invention enhances the evaporation of the solvent and allows the fibrous substance to reach the collector while containing substantially no solvent. The fibrous substance deposited on the collector, therefore, does not become softened and maintains its fibrous shape because of containing substantially no solvent. In addition, a multiplicity of fibers are entangled with each other without lying on the top of one another during flowing by the action of air blowing and are deposited three-dimensionally. Specifically, a solvent drying process and a fiber entangling process can be performed simultaneously. This gives a flocculent three-dimensional structure composed of a fibrous substance having an average diameter of 10 μm or more.

In this connection, another possible solution to the enhancement of the evaporation of the solvent than air blowing is heating, i.e., spinning performed while heating the space between the nozzle and the collector. The present inventors, however, have found that no flocculent three-dimensional structure was obtained by carrying out spinning of a spinning dope as used herein while heating the space between the nozzle and collector at different temperatures. This is probably because the heating does not provide a forced entangling process of fibers, in contrast to the air blowing. This demonstrates that air blowing during spinning is especially advantageous as a technique for enhancing the evaporation of the solvent to give a flocculent three-dimensional structure.

As is described above, the embodiment gives a material for filling bone defects which has a three-dimensional structure composed of a fibrous substance containing a biodegradable resin such as a poly (lactic acid) (PLA) as a principal component and further containing a siloxane, and which has flexibility derived from the three-dimensional structure.

The pore size (pore diameter; void diameter) of such a three-dimensional structure obtained by electrospinning depends on the diameter of the fibrous substance. Specifically, the pore size may decrease with a decreasing fibrous substance diameter and may increase with an increasing fibrous substance diameter. For this reason, the three-dimensional structure according to this embodiment has a pore size larger than that of a corresponding three-dimensional structure composed of a fibrous substance having an average diameter of less than 10 μm.

As is described above, the present inventors have experimentally found that, in the case of a nonwoven fabric, a nonwoven fabric having an average diameter of fibrous substance of 10 μm or more shows higher cell invasion efficiency than one having an average diameter of fibrous substance of less than 10 μm. This indicates that the fibrous substance preferably has an average diameter of 10 μm or more for more satisfactory cell invasion, subsequent growth and proliferation.

Thus, the three-dimensional structure produced according to the embodiment is also expected to show high cell invasion efficiency.

### EXAMPLES

The present invention will be illustrated in further detail with reference to several working examples below which relate to production methods of three-dimensional structures. It should be noted, however, that these examples are illustrated only by way of example for understanding the present invention more deeply and are never intended to limit the scope of the present invention.

Raw Materials Used in Examples

Poly(lactic acid) (PLA): LACEA (Mitsui Chemicals Inc., Japan; weight-average molecular weight Mw: 140 kDa)

Chloroform (CHCl₃): analytical grade reagent, with a purity of 99.0% or more, Kishida Chemical Co., Ltd., Japan

Siloxane-doped calcium carbonate (Si—CaCO₃): Vaterite containing a siloxane in terms of a silicon ion content of 2.9 percent by weight and prepared by using slaked lime (Microstar T; with a purity of 96% or more; Yabashi Industries Co., Ltd., Japan), methanol (analytical grade reagent; with a purity of 99.8% or more; Kishida Chemical Co., Ltd., Japan), APTES (TSL8331, with a purity of 98% or more, GE Toshiba Silicones Co., Ltd., Japan), and carbon dioxide gas (high-purity liquefied carbon dioxide gas; with a purity of 99.9%; Taiyo Kagaku Kogyo K.K., Japan)

Electrospinning Conditions in Examples

Spinning solution feed rate: 0.20 ml/min,

Applied voltage: A voltage was applied to the nozzle at 20 kV, where as the plate collector was grounded,

Distance between the nozzle and the plate collector: 200 mm,

### Example 1

Si—CaCO₃/PLA composites containing 30 and 60 percent by weight of Si—CaCO₃, respectively, were prepared by kneading PLA and Si—CaCO₃ at 180° C. for 10 minutes in a heating kneader. The Si—CaCO₃/PLA composites containing 30 and 60 percent by weight of Si—CaCO₃ are herein after referred to as SiPVH₃₀ and SiPVH₆₀, respectively. The Si—CaCO₃/PLA composites were mixed with chloroform to give spinning dopes. Independently, PLA was mixed with chloroform to give a spinning dope as a referential example. The spinning dopes according to Example 1 and the referential example were prepared so as to have a PLA content of 10 percent by weight relative to chloroform. The PLA dope had a viscosity of 2368 [mPa·s], the SiPVH₃₀ dope had a viscosity of 3986 [mPa·s], and the SiPVH₆₀ had a viscosity of 5312 [mPa·s].

In an electrospinning system as illustrated in FIG. 1, a blower was arranged in a direction perpendicular to the spinning direction (the direction connecting the nozzle with the collector), and a collection box for fiber recovery made of an insulator (expanded polystyrene) was arranged so as to face the blower. Electrospinning of the above-prepared spinning dopes was performed to give Si—CaCO₃/PLA three-dimensional structures. During spinning, air was blown from the blower at a blow rate of about 1 m/s.

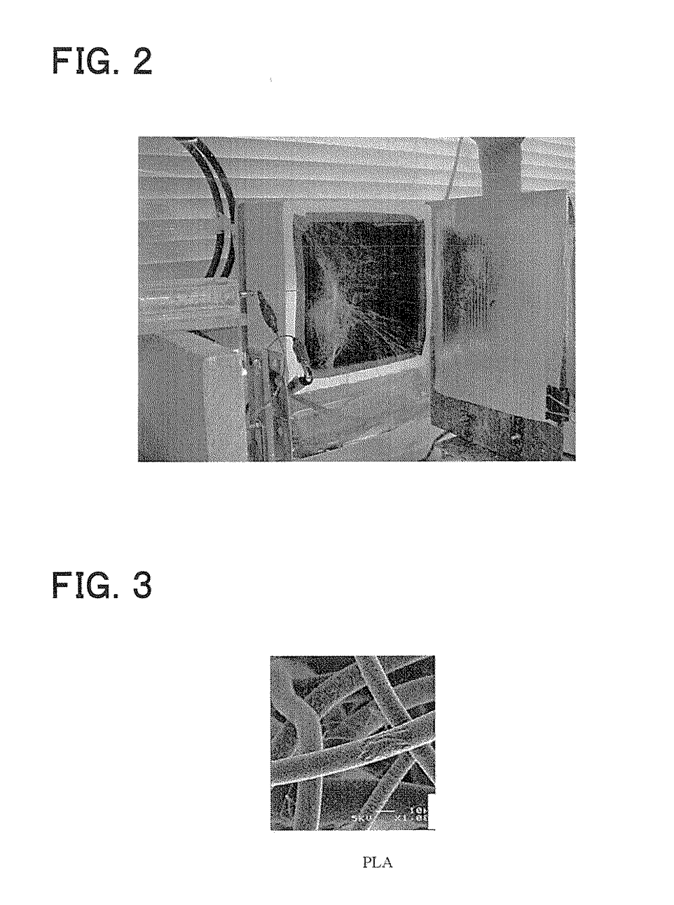

FIG. 2 depicts the appearance of the electrospinning system after spinning. As illustrated in FIG. 2, the spinning with air blowing gave flocculent three-dimensional structures, in which the space between the collection box and the collector was spatially filled with a multiplicity of fibers when any of the spinning dopes was used. This is probably because fibers caught between the collection box and the collector played a role as a kind of collector to give a flocculent three-dimensional structure.

FIG. 3 depicts a scanning electron micrograph (SEM) of the resulting flocculent three-dimensional structure. As is illustrated in FIG. 3, all the spinning dopes gave fibrous substances having an average diameter of 10 μm or more. The average diameter and range of diameters of arbitrary 40 fibers selected from the SEM images were measured. The PLA three-dimensional structure had diameters ranging from 9 to 23 μm with an average diameter of 15 μm; the SiPVH₃₀ three-dimensional structure had diameters ranging from 9 to 32 μm with an average diameter of 18 μm; and the SiPVH₆₀ three-dimensional structure had diameters ranging from 14 to 32 μm with an average diameter of 21 μm.

According to the referential example, the spinning dope containing the PLA alone without siloxane gave a flocculent three-dimensional structure composed of a fibrous substance having an average diameter of 10 μm or more. This indicates that even the spinning dope used in the referential example, if further incorporated with a siloxane, can give a flocculent three-dimensional structure composed of a fibrous substance having an average diameter of 10 μm or more when the viscosity of the spinning dope is suitably set.

What is claimed is:

1. A method for producing a material for filling bone defects, the material having a flocculent three-dimensional structure composed of a fibrous substance containing a biodegradable resin as a principal component and further containing a siloxane, the method comprising the steps of:
dissolving or suspending a substance in a solvent to give a solution or slurry of the substance, the substance containing the biodegradable resin as a principal component and further containing or bearing the siloxane, the solution or slurry having such a viscosity as to form a fibrous substance having an average diameter of 10 μm or more; and
carrying out electrospinning of the solution or slurry,
wherein the electrospinning is performed with air blowing.

2. A material for filling bone defects, the material having a flocculent three-dimensional structure comprising a fibrous substance containing a biodegradable resin as a principal component and further containing a siloxane,
wherein the fibrous substance has an average diameter of 10 μm or more and 100 μm or less.
